(19) United States
(12) Patent Application Publication
(10) Pub. No.: US 2021/0003543 A1
(43) Pub. Date: Jan. 7, 2021

(54) SENSING SYSTEM AND INFORMATION PROCESSING APPARATUS

(71) Applicant: TAIYO YUDEN CO., LTD., Tokyo (JP)

(72) Inventors: Masashi HATTORI, Tokyo (JP); Junji OSHITA, Tokyo (JP); Kenichi SHIMOMAI, Tokyo (JP)

(73) Assignee: TAIYO YUDEN CO., LTD., Tokyo (JP)

(21) Appl. No.: 17/028,040

(22) Filed: Sep. 22, 2020

Related U.S. Application Data
(63) Continuation of application No. PCT/JP2019/004296, filed on Feb. 6, 2019.

(30) Foreign Application Priority Data
Mar. 29, 2018 (JP) 2018-066145

(51) Int. Cl. G01N 33/00; G01N 25/48

(52) U.S. Cl. CPC G01N 33/0031; G01N 33/0062; G01N 2033/0068; G01N 33/0073; G01N 25/48

(57) ABSTRACT

A sensing system includes: a first odor sensor including one or more detection elements respectively detecting an amount of an odor-causing substance or substances existing in air; a filter to remove the odor-causing substance existing in air; a second odor sensor including one or more detection elements respectively detecting an amount of the odor-causing substance or substances existing in air having passed through the filter; and one or more processor generating and outputting one or more detection signals by calculating a difference between one or more first output signals generated by the one or more detection elements of the first odor sensor and one or more second output signals generated by the one or more detection elements of the second odor sensor, the one or more processors generating and outputting odor information based on one or more waveforms of the generated one or more detection signals.

### FIELD

The present invention relates generally to a sensing system and an information processing apparatus.

### BACKGROUND

In recent years, odor sensor elements have extensively been developed. Known as an odor sensor element is, for example, a quartz crystal microbalance (QCM) sensor in which a film adsorbing an odor-causing substance is provided on the surface of a quartz oscillator. An AT-cut quartz oscillator changes in resonance frequency by a mass change. The QCM sensor detects a change amount of the resonance frequency by oscillating the AT-cut quartz crystal, and thereby detects the mass of a causing substance.

Also known is a sensor apparatus including a plurality of odor sensor elements detecting the masses of respective different causing substances. Such a sensor apparatus is able to output the mass of each of the causing substances. An information processing apparatus receives the amount of each of the causing substances output from such a sensor apparatus and compares a pattern of the amount of each of the received causing substances with patterns recorded in advance. Accordingly, the information processing apparatus is able to identify the type of odor.

Such a sensor apparatus is able to be applied to a system that collects many types of odors and stores the collected odors in a database. By collecting many types of odors and analyzing the many types of collected odors, it is possible to handle, as quantitative information, odors that have been individually identified by a sense of human beings so far.

By the way, the sensor apparatus including the odor sensor elements has a complicated circuit configuration, and its detection signals contain various kinds of noise.

Thus, when many kinds of odors are collected, information contains a lot of noise. Further, when monitoring odors, detection signals representing no odor need to be recorded, so that information cannot be collected efficiently.

### SUMMARY

A sensing system according to the present invention includes: a first odor sensor including one or more detection elements respectively configured to detect an amount or amounts of an odor-causing substance or odor-causing substances existing in air, the one or more detection elements respectively outputting one or more first output signals; a filter configured to remove the odor-causing substance existing in air; a second odor sensor including one or more detection elements respectively configured to detect an amount or amounts of the odor-causing substance or substances existing in air that has passed through the filter, the one or more detection elements respectively outputting one or more second output signals; and one or more processors that generate one or more detection signals by calculating a difference between the one or more first output signals from the one or more detection elements of the first odor sensor and the one or more second output signals from the one or more detection elements of the second odor sensor, the one or more processors generating odor information representing at least one of characteristics of the one or more detection signals based on one or more waveforms of the one or more detection signals and outputting the generated odor information.

According to the present invention, information regarding odors can be collected with high accuracy and high efficiency.

### BRIEF DESCRIPTION OF THE DRAWINGS

FIG. 1 is a diagram illustrating a configuration of a sensing system;

FIG. 2 is a diagram illustrating an exemplary configuration of a sensor unit;

FIG. 3 is a diagram illustrating an exemplary configuration of a first odor sensor;

FIG. 4 is a diagram illustrating a functional configuration of a signal processing unit;

FIG. 5 is a chart illustrating exemplary waveforms of a plurality of first output signals output from the first odor sensor;

FIG. 6 is a chart illustrating exemplary waveforms of a plurality of second output signals output from a second odor sensor;

FIG. 7 is a chart illustrating exemplary waveforms of a plurality of detection signals output from a difference calculation unit;

FIG. 8 is a diagram illustrating a functional configuration of an information collection unit;

FIG. 9 is a diagram for explaining odor determination processing;

FIG. 10 is a chart illustrating examples of a plurality of waveforms of detection signals, a start timing, and an end timing;

FIG. 11 is a table illustrating exemplary information recorded in an information storage unit;

FIG. 12 is a flowchart illustrating a procedure of processing of the information collection unit;

FIG. 13 is a diagram illustrating a configuration of a sensing system according to a modification;

FIG. 14 is a flowchart illustrating a procedure of processing of an information collection unit according to the modification; and

FIG. 15 is a diagram illustrating a hardware configuration of an information processing apparatus.

### DETAILED DESCRIPTION

Hereinafter, a sensing system 10 according to an embodiment will be described with reference to the accompanying drawings. The sensing system 10 continuously monitors odors and registers odor information about the odors acquired by monitoring.

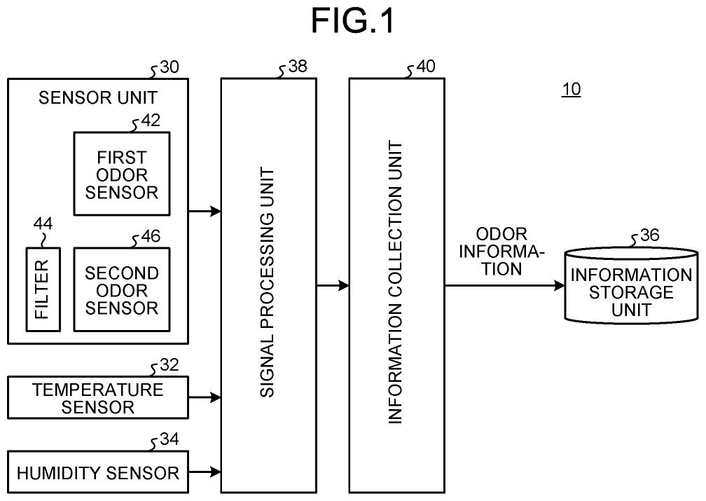

FIG. 1 is a diagram illustrating a configuration of the sensing system 10. The sensing system 10 includes a sensor unit 30, a temperature sensor 32, a humidity sensor 34, an information storage unit 36, a signal processing unit 38, and an information collection unit 40.

The sensor unit 30 includes a first odor sensor 42, a filter 44, and a second odor sensor 46.

The first odor sensor 42 includes one or more detection elements each detecting an amount of an odor-causing substance existing in air. Each of the detection elements detects a mass of a causing substance as the amount of the odor-causing substance. In place thereof, each of the detection elements may detect the volume or the molecular weight of the causing substance as the amount of the causing substance.

In a case where the first odor sensor 42 includes a plurality of detection elements, those detection elements are of different types from each other. For example, any two of the detection elements included in the first odor sensor 42 detect the amounts of different types of odor-causing substances. In this case, while a first detection element detects the amount of a substance X, a second detection element detects the amount of a substance Y. Furthermore, any two detection elements included in the first odor sensor 42 may detect the amounts of the same type of odor-causing substance with different sensitivities, for example. In this case, while the first detection element detects the amount of the substance X with a first sensitivity, the second detection element detects the amount of the substance X with a second sensitivity that is lower than the first sensitivity.

Any two detection elements included in the first odor sensor 42 may detect the amounts of a plurality of odor-causing substances in combinations of different types, for example. In this case, while a first detection element detects the total amount of the substance X and the substance Y, a second detection element detects the total amount of the substance X and a substance Z, for example. Any two detection elements included in the first odor sensor 42 may detect the amounts of a plurality of odor-causing substances in combinations of the same types with different sensitivities, for example. In this case, while the first detection element may detect the total amount of the substance X and the substance Y with the first sensitivity, the second detection element may detect the total amount of the substance X and the substance Y with the second sensitivity that is lower than the first sensitivity, for example.

The filter 44 is provided in a preceding stage of the second odor sensor 46. The filter 44 removes the odor-causing substance existing in air to be given to the second odor sensor 46.

The second odor sensor 46 includes one or more detection elements each detecting the amount of the odor-causing substance existing in air having passed through the filter 44. The second odor sensor 46 has the same configuration as that of the first odor sensor 42. Therefore, the first odor sensor 42 and the second odor sensor 46 include one or more detection elements of the same types.

The sensor unit 30 transmits, to the signal processing unit 38, one or more first output signals detected by the one or more detection elements included in the first odor sensor 42. The sensor unit 30 also transmits, to the signal processing unit 38, one or more second output signals detected by the one or more detection elements included in the second odor sensor 46.

The first odor sensor 42, the filter 44, and the second odor sensor 46 are disposed near one another. The first odor sensor 42 and the second odor sensor 46 may be disposed within a same casing or may be provided independently from each other.

The temperature sensor 32 is provided near the first odor sensor 42 and the second odor sensor 46. The temperature sensor 32 detects ambient temperatures of the first odor sensor 42 and the second odor sensor 46. The temperature sensor 32 transmits, to the signal processing unit 38, temperature signals representing the ambient temperatures of the first odor sensor 42 and the second odor sensor 46.

The humidity sensor 34 is provided near the first odor sensor 42 and the second odor sensor 46. The humidity sensor 34 detects ambient humidity of the first odor sensor 42 and the second odor sensor 46. The humidity sensor 34 transmits, to the signal processing unit 38, humidity signals representing the ambient humidity of the first odor sensor 42 and the second odor sensor 46.

The information storage unit 36 stores odor information for each odor.

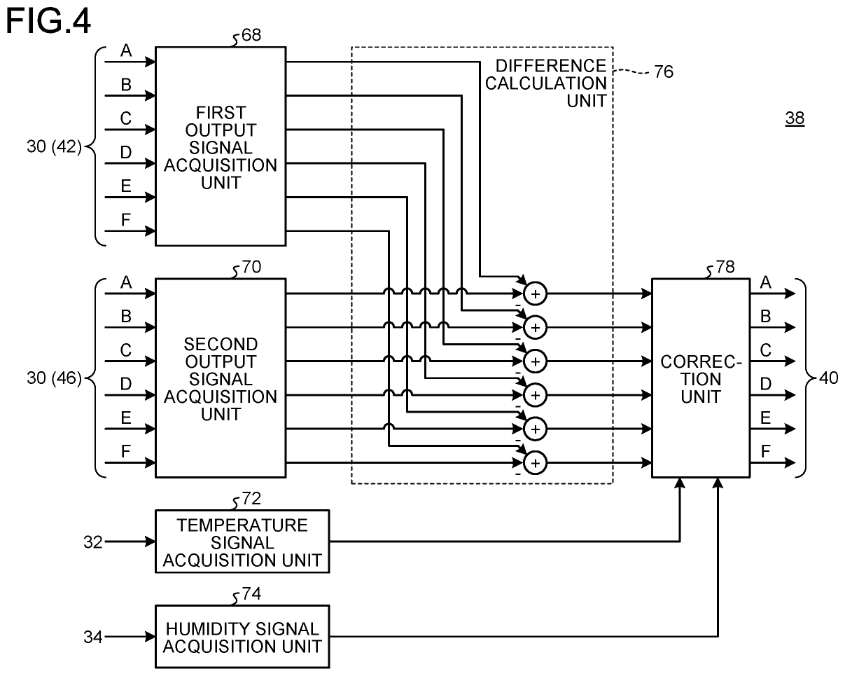

The signal processing unit 38 generates one or more detection signals by calculating a difference between the one or more first output signals detected by the one or more detection elements of the first odor sensor 42 and the one or more second output signals detected by the one or more detection elements of the second odor sensor 46. The signal processing unit 38 calculates differences between the first output signals and the second outputs signals of the same type of detection elements, thereby generating detection signals. Furthermore, the signal processing unit 38 corrects one or more detection signals based on a temperature signal transmitted from the temperature sensor 32 and a humidity signal transmitted from the humidity sensor 34. The signal processing unit 38 transmits the one or more detection signals to the information collection unit 40. Note that the signal processing unit 38 will be described in more detail with referring to FIG. 4.

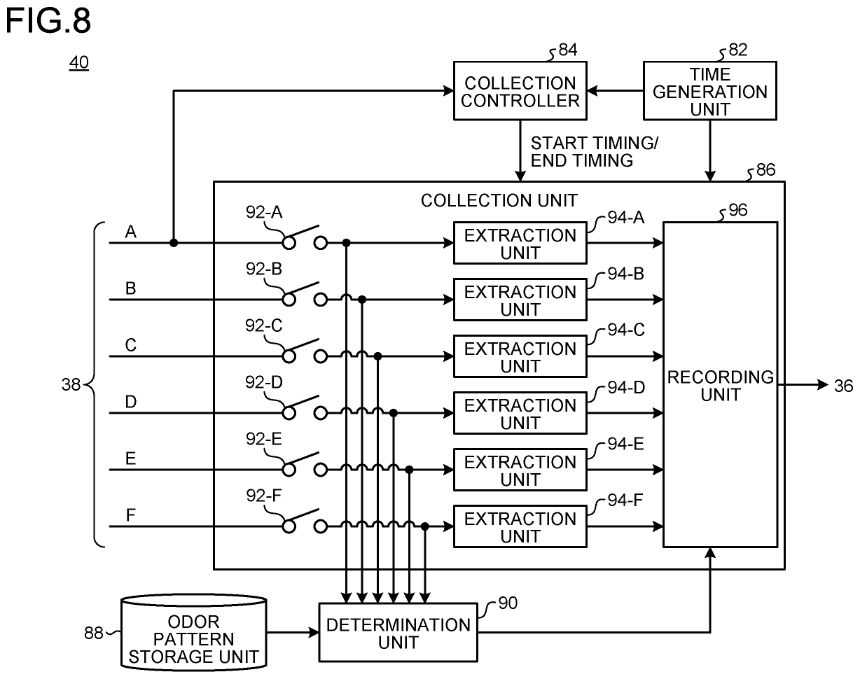

When an odor occurs in air, the information collection unit 40 generates odor information regarding the odor occurred in air based on one or more detection signals transmitted from the signal processing unit 38. The information collection unit 40 loads the generated odor information into the information storage unit 36. Note that the information collection unit 40 will be described in more detail with referring to FIG. 8.

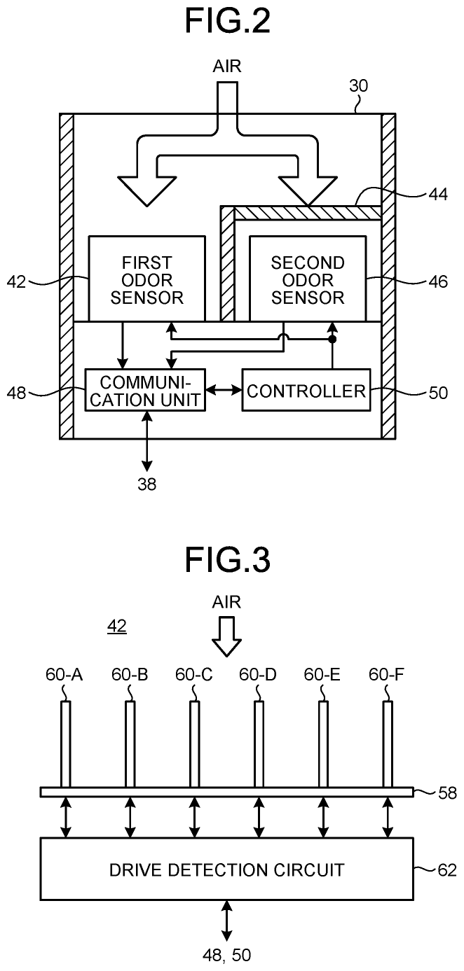

FIG. 2 is a diagram illustrating an exemplary configuration of the sensor unit 30. The sensor unit 30 includes the first odor sensor 42, the filter 44, the second odor sensor 46, a communication unit 48, and a controller 50.

The first odor sensor 42 and the second odor sensor 46 are disposed within a same casing, for example. Air that has not passed through the filter 44 is given to the first odor sensor 42. The first odor sensor 42 outputs the one or more first output signals representing an odor (or odors) in air having not passed through the filter 44. Air that has passed through the filter 44 is given to the second odor sensor 46. The second odor sensor 46 outputs the one or more second output signals representing an odor (or odors) in air having passed through the filter 44.

The communication unit 48 transmits, to the signal processing unit 38, the one or more first output signals output from the first odor sensor 42. The communication unit 48 also transmits, to the signal processing unit 38, the one or more second output signals output from the second odor sensor 46.

The controller 50 manages and controls operation of the first odor sensor 42, the second odor sensor 46, and the communication unit 48. Note that the configuration of the sensor unit 30 is an example, and any configuration may be employed.

FIG. 3 is a diagram illustrating an exemplary configuration of the first odor sensor 42. In the present embodiment, the first odor sensor 42 is a quartz crystal microbalance (QCM) sensor that is capable of detecting the mass of minute substances contained in air. The first odor sensor 42 is not limited to the QCM sensor but may be another type of sensor such as a gas sensor using a semiconductor thin film. While the exemplary configuration of the first odor sensor 42 is described, the second odor sensor 46 has the same configuration as that of the first odor sensor 42.

The first odor sensor 42 includes, as a way of example, a support member 58, a plurality of gas detection elements 60, and a drive detection circuit 62. The gas detection elements 60 are attached to the support member 58.

The gas detection elements 60 are an example of the detection elements. In the example in FIG. 3, the first odor sensor 42 includes six different types of gas detection elements 60-A to 60-F. The six gas detection elements 60-A to 60-F detect respective different types of odor-causing substances.

Each of the gas detection elements 60 includes: a quartz oscillator whose body is cut in a shape capable of oscillating by the piezoelectric effect; two electrodes provided on planes of both sides of the quartz oscillator; and an adsorption film provided on at least one of the planes of the quartz oscillator.

Part of a side face of the quartz oscillator is held by the support member 58 so as to allow oscillating. An AC voltage is applied from the drive detection circuit 62 to the two electrodes. The adsorption film adsorbs a specific causing substance existing in the ambient air. Each of the gas detection elements 60 includes an adsorption film adsorbing a substance different from each other. Specifically, each of the gas detection elements 60 includes the adsorption film that adsorbs a causing substance as an object to be detected by the sensor unit 30.

In the gas detection element 60, when an AC voltage with a resonance frequency is applied to the two electrodes, the quartz oscillator oscillates by the piezoelectric effect. The fundamental resonance frequency of the quartz oscillator is determined by mass and viscoelasticity. Consequently, when the adsorption film adsorbs the causing substance thereby changing the mass, the fundamental resonance frequency of the gas detection element 60 changes in accordance with the change in the mass of the adsorbed substance.

The drive detection circuit 62 applies an AC voltage to each of the gas detection elements 60 in accordance with control of the controller 50, and detects a change in the fundamental resonance frequency of each of the gas detection elements 60. Thus, the drive detection circuit 62 is able to detect, for each of the gas detection elements 60, the mass of the odor-causing substance contained in the given air. The drive detection circuit 62 gives the first output signals representing the masses of the causing substances detected by each of the gas detection elements 60 to the communication unit 48.

FIG. 4 is a diagram illustrating a functional configuration of the signal processing unit 38. The signal processing unit 38 includes a first output signal acquisition unit 68, a second output signal acquisition unit 70, a temperature signal acquisition unit 72, a humidity signal acquisition unit 74, a difference calculation unit 76, and a correction unit 78.

The first output signal acquisition unit 68 acquires one or more first output signals detected by the one or more detection elements included in the first odor sensor 42 of the sensor unit 30. The second output signal acquisition unit 70 acquires one or more second output signals detected by the one or more detection elements included in the second odor sensor 46 of the sensor unit 30.

The temperature signal acquisition unit 72 acquires a temperature signal output from the temperature sensor 32. The humidity signal acquisition unit 74 acquires a humidity signal output from the humidity sensor 34.

The difference calculation unit 76 generates one or more detection signals by calculating a difference between: the one or more first output signals detected by the one or more detection elements included in the first odor sensor 42; and the one or more second output signals detected by the one or more detection elements included in the second odor sensor 46. The difference calculation unit 76 calculates the difference between the first output signal and the second output signal of the detection elements of the same type.

In the present embodiment, the difference calculation unit 76 calculates differences between: six first output signals detected by the six gas detection elements 60-A to 60-F included in the first odor sensor 42; and six second output signals detected by the six gas detection elements 60-A to 60-F included in the second odor sensor 46. Then, the difference calculation unit 76 outputs six detection signals corresponding to each of the six gas detection elements 60-A to 60-F.

The correction unit 78 corrects one or more detection signals generated by the difference calculation unit 76 based on the temperature of the ambient air of the first odor sensor 42 and the second odor sensor 46 represented by the temperature signal. Furthermore, the correction unit 78 corrects one or more detection signals generated by the difference calculation unit 76 based on the humidity of the ambient air of the first odor sensor 42 and the second odor sensor 46 represented by the humidity signal. Note that the correction unit 78 may correct the one or more detection signals generated by the difference calculation unit 76 based on one of the temperature and humidity.

As for circuits and materials included in the first odor sensor 42 and the second odor sensor 46, properties thereof change depending on the ambient temperature and humidity. Thus, the one or more detection signals generated by the difference calculation unit 76 change depending on the temperature and humidity. In order to eliminate such variation in values depending on the temperature and humidity, the correction unit 78 corrects the one or more detection signals generated by the difference calculation unit 76 in accordance with the detected temperature and humidity.

The correction unit 78 may correct the signals in a preceding stage of the difference calculation unit 76. That is to say, the correction unit 78 may correct the first output signals and the second output signals in accordance with the temperature and humidity. With this configuration, the correction unit 78 is also possible to acquire the same effect as that acquired by correcting the one or more signals generated by the difference calculation unit 76.

The signal processing unit 38 transmits the one or more generated detection signals to the information collection unit 40. In the present embodiment, the signal processing unit 38 transmits six detection signals corresponding to the six gas detection elements 60-A to 60-F to the information collection unit 40.

The signal processing unit 38 may be implemented by a digital processing circuit or an analog processing circuit. The signal processing unit 38 may also be implemented by a processor executing a computer program and a memory.

Furthermore, the signal processing unit 38 may be provided integrally in the sensor unit 30. As for the signal processing unit 38, either one of the difference calculation unit 76 and the correction unit 78 may be provided in the sensor unit 30, while the other is provided integrally with the information collection unit 40 in a post-stage.

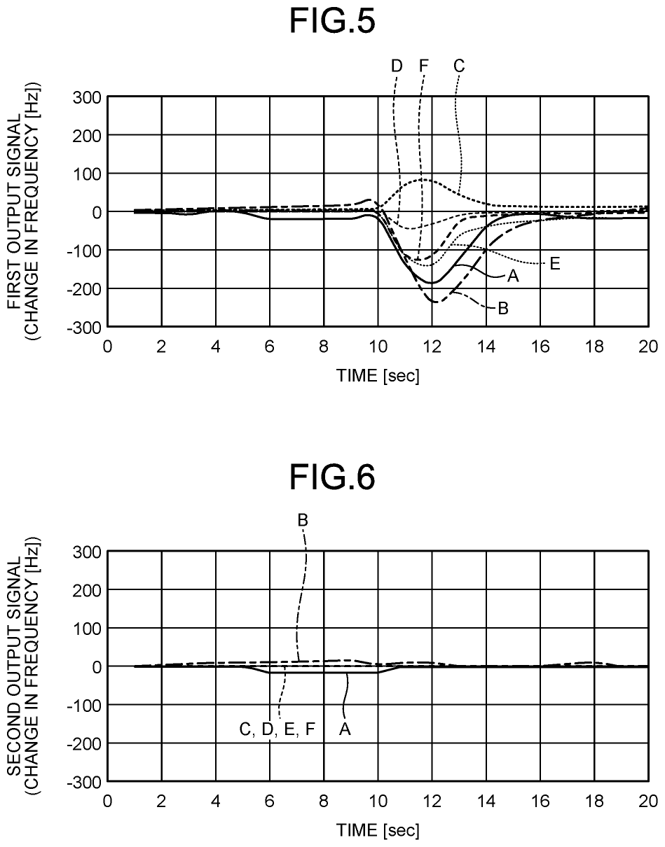

FIG. 5 is a chart illustrating exemplary waveforms of a plurality of first output signals output from the first odor sensor 42. In FIG. 5, the horizontal axis represents time, and the longitudinal axis represents values of the first output signals (change amount of frequency).

When an odor occurs in the surroundings of the sensor unit 30, the six gas detection elements 60-A to 60-F included in the first odor sensor 42 output the first output signals as illustrated in FIG. 5, for example. From the waveforms of the six first output signals in FIG. 5, it can be seen that odors start to occur at the point of about 10 seconds and end (that is, disappear) at the point of about 16 seconds. The six first output signals gradually increase or decrease from the odor start point, reach the peaks (maximal or minimal), then gradually decrease or increase and return to original values at the point where the odors disappear. The six first output signals form waveforms different from each other, and the increase or decrease rates and the values at the peaks thereof are different. Thus, the sensing system 10 is able to quantify the odors by analyzing respective characteristics of such six signal waveforms and mutual relations of the six signal waveforms.

FIG. 6 is a chart illustrating exemplary waveforms of a plurality of second output signals output from the second odor sensor 46. In FIG. 6, the horizontal axis represents time, and the longitudinal axis represents values of the second output signals (change amount of frequency).

When an odor occurs in the surroundings of the sensor unit 30, the six gas detection elements 60-A to 60-F included in the second odor sensor 46 output the second output signals as illustrated in FIG. 6, for example.

The six gas detection elements 60-A to 60-F included in the second odor sensor 46 detect the amounts of odor-causing substances existing in air from which the odor-causing substances have been removed by the filter 44. Thus, even if odors occur, there is no change in the values of the six second output signals due to the odors.

However, the first output signals and the second output signals contain various kinds of noise due to causes other than odors. For example, the first output signals and the second output signals illustrated in FIG. 5 and FIG. 6 contain common-mode noise in a period from about 5 seconds to about 11 seconds.

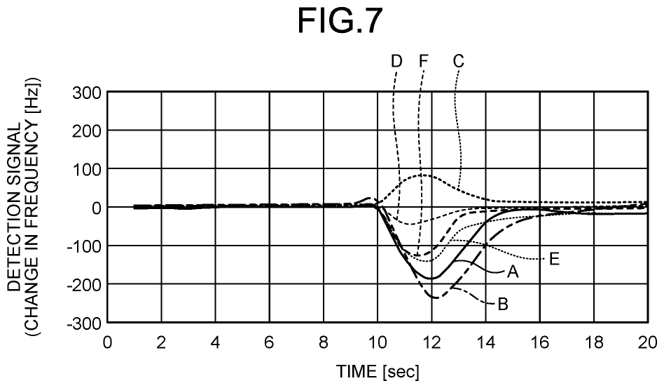

FIG. 7 is a chart illustrating exemplary waveforms of a plurality of detection signals output from the difference calculation unit 76. In FIG. 7, the horizontal axis represents time, and the longitudinal axis represents values of the detection signals (change amount of frequency).

The difference calculation unit 76 subtracts the six second output signals output from the second odor sensor 46 from the six first output signals output from the first odor sensor 42, and outputs six detection signals.

Thereby, in the detection signals, the commo-mode noise contained in the first output signals and the second output signals is offset and fluctuation components caused by the odors contained in the first output signals remain. For example, as in FIG. 7, as for the detection signals outputted from the difference calculation unit 76, noise generated in the period from about 5 seconds to 11 seconds is removed. Thus, the difference calculation unit 76 is able to output, with high accuracy, the detection signals representing changes caused by occurrence of the odors.

FIG. 8 is a diagram illustrating a functional configuration of the information collection unit 40. The information collection unit 40 includes a time generation unit 82, a collection controller 84, a collection unit 86, an odor pattern storage unit 88, and a determination unit 90.

The time generation unit 82 generates time information. The time generation unit 82 may generate count values acquired by counting a clock or the like from a reference time (for example, operation start time). The time generation unit 82 gives the generated time information to the collection controller 84 and the collection unit 86.

The collection controller 84 detects a start timing at which an odor starts to occur and an end timing at which the odor disappears. The collection controller 84 notifies the collection unit 86 of the detected start timing and end timing.

The collection controller 84 detects, as the start timing, a timing at which one or more detection signals start to change. For example, the collection controller 84 acquires any one of the one or more detection signals. Then, the collection controller 84 detects, as the start timing, a timing at which the one acquired detection signal changes more greatly than a predetermined threshold value.

The threshold value is arbitrarily set by a user, for example. The threshold value is determined based on, for example, the environment where the sensing system 10 is placed and a predicted value of the intensity of the odor to be caused. Furthermore, for example, the collection controller 84 may acquire two or more detection signals among a plurality of detection signals, and detect, as the start timing, a timing at which an added value or an average value of the two or more detection signals changes more greatly than a predetermined threshold value.

The collection controller 84 detects, as the end timing, a timing that has elapsed a given time (for example, n seconds later (n is a positive value)) from the start timing. Furthermore, the collection controller 84 may detect, as the end timing, a timing at which the change in the one or more detection signal ends. For example, the collection controller 84 acquires any one of the one or more detection signals. Then, the collection controller 84 may detect, as the end timing, a timing at which the value of the one acquired detection signal returns to the original value after the start timing.

The collection unit 86 collects, as odor information, the information generated based on the one or more detection signals received from the signal processing unit 38. More specifically, the collection unit 86 extracts the waveform corresponding to a period from the start timing to the end timing in the one or more detection signals received from the signal processing unit 38. Then, the collection unit 86 collects, as odor information, information generated based on the extracted waveform and information related to the extracted waveform.

For example, the collection unit 86 collects, as the odor information, information representing the waveform of the one or more detection signals in a given period from the start timing. Thereby, the collection unit 86 is able to collect information representing the waveform of the detection signal in the period where the odor is present.

Furthermore, for example, the collection unit 86 collects, as the odor information, a feature amount acquired from the waveform of the one or more detection signals in a given period from the start timing. Thereby, the collection unit 86 is able to collect the feature amount of the waveform of the detection signal in the period where the odor is present.

Furthermore, for example, the collection unit 86 collects, as the odor information, a change amount of the one or more detection signals from the start timing to a first time later (for example, m seconds later; m is a positive value). Thereby, the collection unit 86 is able to collect, as one of features of the waveform of the detection signal in the period where the odor is present, a changing rate of the intensity of the odor at the odor start point.

Furthermore, for example, the collection unit 86 collects, as the odor information, a maximal value and an minimal value of the one or more detection signals from the start timing to a second time later (for example, n seconds later; n is a positive value). The maximal value is a value of a detection signal at the point where the detection signal reaches the peak of an upward convex. The minimal value is a value of a detection signal at the point where the detection signal reaches the peak of a downward convex. Thereby, the collection unit 86 is able to collect, as one of features of the waveform of the detection signal in the period where the odor is present, the maximum amplitude of the intensity of the odor.

Furthermore, the collection unit 86 collects, as odor information, the type of odor determined based on the one or more detection signals in a given period from the start timing. The collection unit 86 acquires the type of odor from the determination unit 90 to be described later. Thereby, the collection unit 86 is able to collect the type of the generated odor.

Furthermore, for example, the collection unit 86 collects the time of the start timing as odor information. Thereby, the collection unit 86 is able to collect the time at which the odor occurs.

The collection unit 86 stores the odor information collected in the manner described above in the information storage unit 36. For example, the collection unit 86 stores the odor information regarding the odor in the information storage unit 36 for each of the generated odors.

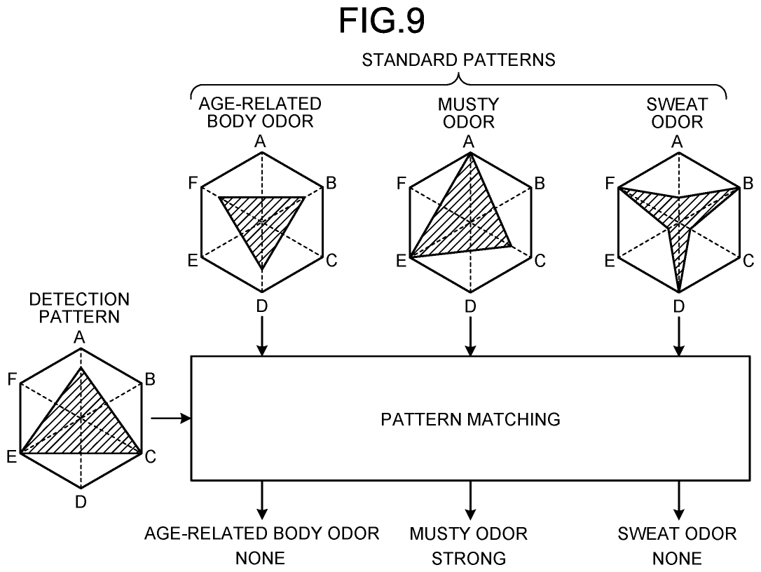

The odor pattern storage unit 88 stores one or more standard patterns in association with one or more types of odors for each pattern. The determination unit 90 acquires, from the collection unit 86, one or more detection values at any timings between the start timing and the end timing. For example, the determination unit 90 acquires the value of one or more detection signals at a point that is a specific time later (for example, the first time later (m seconds later)) from the start timing. The determination unit 90 determines the odor based on the acquired value of the one or more detection signals. The determination unit 90 gives a determination result to the collection unit 86. Note that determination processing will further be described by referring to FIG. 9.

In the present embodiment, the collection unit 86 includes six switches 92-A to 92-F, six extraction units 94-A to 94-F, and a recording unit 96. The six switches 92-A to 92-F correspond one-to-one to the six gas detection elements 60-A to 60-F. Each of the six switches 92-A to 92-F allows the corresponding detection signals to pass through in a period from the start timing to the end timing, and blocks the signals in other periods.

The six extraction units 94-A to 94-F correspond one-to-one to the six gas detection elements 60-A to 60-F. Each of the six extraction units 94-A to 94-F receives the corresponding detection signal that has passed through the switch 94. Each of the six extraction units 94-A to 94-F extracts, as a first feature amount, a change amount in a period from the start timing to a given first time later (for example, m seconds later) in a waveform of the received detection signal. Furthermore, each of the six extraction units 94-A to 94-F extracts, as a second feature amount, a maximal value or a minimal value in a period from the start timing to a given second time later (for example, n seconds later) in a waveform of the received detection signal.

The recording unit 96 acquires the time of the start timing and acquires the first feature amounts and the second feature amounts extracted by each of the six extraction units 94-A to 94-F. Furthermore, the recording unit 96 acquires the type of odor determined by the determination unit 90 and information representing the waveform of the one or more detection signals in a period from the start timing to the end timing. Then, the recording unit 96 records the acquired information in the information storage unit 36 as odor information.

FIG. 9 is a diagram for explaining the odor determination processing. The determination unit 90 acquires the value of the one or more detection signals at a specific time from the start timing (for example, the first time later (m seconds later), for example. In the example of FIG. 9, for example, the determination unit 90 acquires the values of the six detection signals corresponding to each of the gas detection elements 60-A, the gas detection elements 60-B, the gas detection elements 60-C, the gas detection elements 60-D, the gas detection elements 60-E, and the gas detection elements 60-F.

The odor pattern storage unit 88 stores a standard pattern representing the value of the one or more detection signals acquired when a given type of odor occurs. For example, in the example in FIG. 9, the odor pattern storage unit 88 stores standard patterns representing the values of the six detection signals corresponding to each of the gas detection elements 60-A to 60-F acquired when each of an age-related body odor, a musty odor, and a sweat odor is detected.

The determination unit 90 compares a detection pattern representing the value of the one or more detection signals with the standard patterns stored in advance. Then, when the detection pattern matches the standard pattern, the determination unit 90 determines that the odor of air given to the sensor unit 30 is a given type of odor. The determination unit 90 may store in advance the standard patterns for a plurality of types of odors, and determine which of the odor standard patterns matches a detection pattern. For example, in the example in FIG. 9, the determination unit 90 determines that the odor in air given to the sensor unit 30 is a musty odor.

The case where patterns match each other refers to not only a case where two patterns completely match each other but also a case where they match each other with a given error or less, a case where the closest standard pattern out of a plurality of standard patterns is selected, and the like.

The determination unit 90 may determine the intensity of odor for each type of odor. For example, the determination unit 90 may store standard pattern for each type of odor and intensity of odor in advance, and perform matching between a detection pattern and the standard patterns for each type of odor and intensity of odor stored in advance.

The determination unit 90 may determine the type of odor and the intensity of odor by not only such pattern matching but also another method. For example, the determination unit 90 may determine the type of odor and the intensity of odor matching the detection pattern by using a neural network or the like.

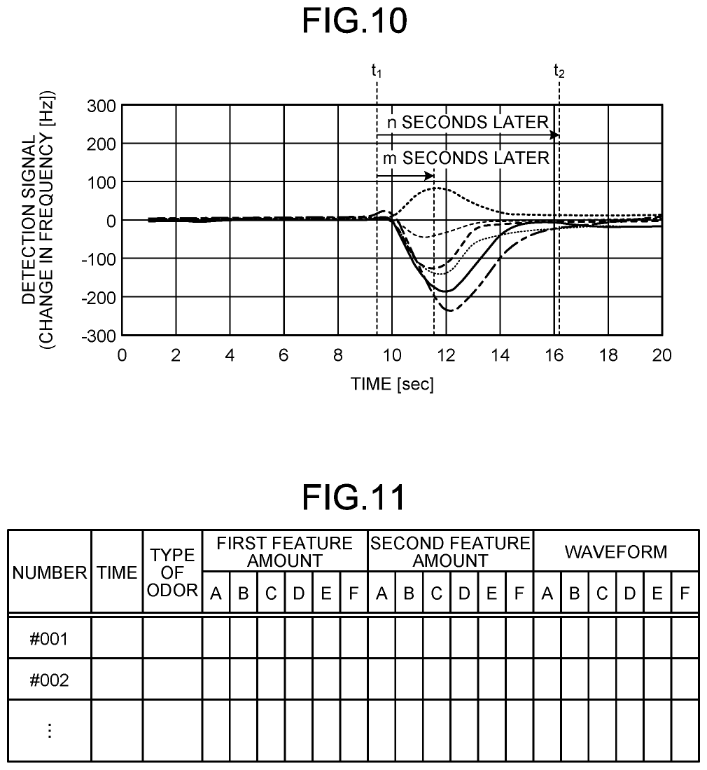

FIG. 10 is a chart illustrating examples of the waveforms of a plurality of detection signals, the start timing, and the end timing. In FIG. 10, the horizontal axis represents time, and the longitudinal axis represents values of the detection signals (change amount of frequency).

The collection controller 84 detects, as the start timing, time t₁ at which the change of the detection signals starts. Furthermore, the collection controller 84 detects, as the end timing, time t₂ that is n seconds later from the start timing (the time t₁).

In such a case, the collection unit 86 collects the waveforms of the six detection signals from the time t₁ to the time t₂. Furthermore, the collection unit 86 collects, as the first feature amount, the change amount from the start timing (the time t₁) to m seconds later in each of the six detection signals. Furthermore, the collection unit 86 collects, as the second feature amount, the maximal value or the minimal value in a period from the start timing (the time t₁) to n seconds later (the second time later) in each of the six detection signals. Furthermore, the collection unit 86 collects the time t₁ of the start timing, for example.

FIG. 11 is a table illustrating exemplary information recorded in the information storage unit 36. The information storage unit 36 stores, for each of the generated odors, the odor information collected by the collection unit 86.

The information storage unit 36 stores the table as illustrated in FIG. 11. The table stores, for each of the generated odors, the unique number, the generation time of odor, the type of odor, the first feature amount, the second feature amount, and information representing the waveform. The table stores the first feature amount, the second feature amount, and the information representing the waveform for each of the detection elements (for example, each of the six gas detection elements 60-A to 60-F) included in the first odor sensor 42 (the second odor sensor 46).

Note that the information storage unit 36 may not store the waveforms of the detection signals. This makes it possible to reduce the information amount to be stored in the information storage unit 36. Furthermore, the collection unit 86 may collect other types of feature amounts than the first feature amount and the second feature amount and store those in the information storage unit 36.

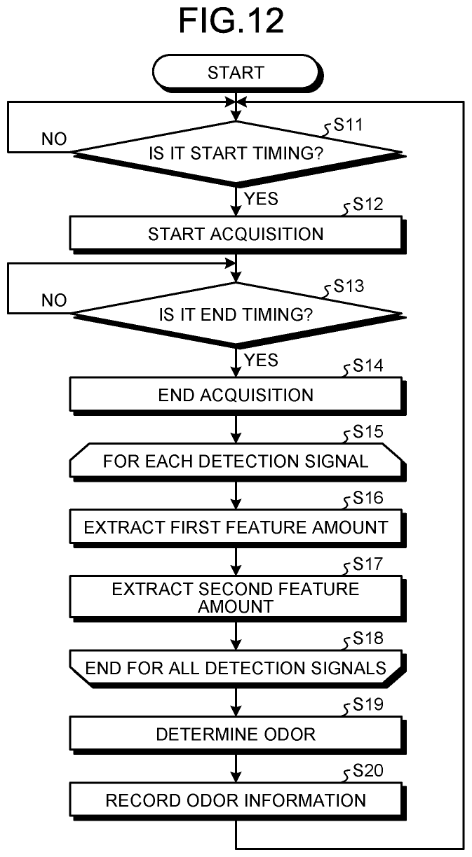

FIG. 12 is a flowchart illustrating a procedure of processing of the information collection unit 40. The information collection unit 40 executes processing in FIG. 12, for example.

At S11, the information collection unit 40 determines whether it is the start timing. For example, the information collection unit 40 determines whether any one of the detection signals has changed more greatly than the predetermined threshold value. When it is not the start timing (No at S11), the information collection unit 40 stands by for the process at S11. When it is the start timing (Yes at S11), the information collection unit 40 advances the process to S12.

At S12, the information collection unit 40 starts acquisition processing of one or more detection signals. For example, the information collection unit 40 starts recording of waveform data of the one or more detection signals.

Subsequently, at S13, the information collection unit 40 determines whether it is the end timing. For example, the information collection unit 40 determines whether a given time has elapsed from the start timing. When it is not the end timing (No at S13), the information collection unit 40 stands by for the process at S13. When it is the end timing (Yes at S13), the information collection unit 40 advances the process to S14.

At S14, the information collection unit 40 ends the acquisition processing of the one or more detection signals. For example, the information collection unit 40 ends the recording of the waveform data of the one or more detection signals.

Subsequently, in a loop of the processes of S15 to S18, the information collection unit 40 executes the processes of S16 and S17 for each of the detection signals. At S16, the information collection unit 40 extracts the first feature amount from the fetched processing-target detection signal. Subsequently, at S17, the information collection unit 40 extracts the second feature amount from the fetched processing-target detection signal. When the first feature amount and the second feature amount are extracted for all of the detection signals, the information collection unit 40 advances the process to S19.

At S19, the information collection unit 40 determines the type of the generated odor based on the one or more acquired detection signals. For example, the information collection unit 40 determines the type of the odor based on the value of the one or more detection signals at a point that is a specific time later (for example, the first time later (m seconds later)) from the start timing.

Subsequently, at S20, the information collection unit 40 records the odor information in the information storage unit 36. Specifically, the information collection unit 40 records, in the information storage unit 36, the time of the start timing, the type of the odor, the first feature amount, the second feature amount, and the information representing the waveform. When the process of S20 ends, the information collection unit 40 returns the process to S11 and repeats the process from S11.

The sensing system 10 according to the present embodiment as described above is able to monitor the odor and collect the information regarding the generated odor. Thereby, the sensing system 10 makes it possible to handle, as quantitative information based on the collected odor information, odors that are used to be determined by a sense of human beings.

Furthermore, the sensing system 10 is able to collect the odor information based on a difference between a detection result of the amount of the odor-causing substance existing in air having passed through the filter 44 and a detection result of the amount of the odor-causing substance existing in air having not passed through the filter 44. Thereby, the sensing system 10 makes it possible to remove the noise and collect the odor information with high accuracy.

Furthermore, the sensing system 10 is capable of monitoring the odor and collecting the waveform of the detection signal in the section where the odor is present and the feature amounts of the waveform as the odor information. Therefore, the sensing system 10 is able to efficiently collect the information that is effective for analyzing the odor.

As described above, the sensing system 10 according to the present embodiment makes it possible to collect the information regarding the odor with high accuracy and high efficiency.

Modification

Next, the sensing system 10 according to a modification will be described. The sensing system 10 according to the modification has substantially the same functions and structures as those of the sensing system 10 according to the present embodiment described with reference to FIG. 1 to FIG. 12. Thus, same reference signs are applied to the blocks of substantially the same functions and structures and detailed explanations are not provided except for the different points.

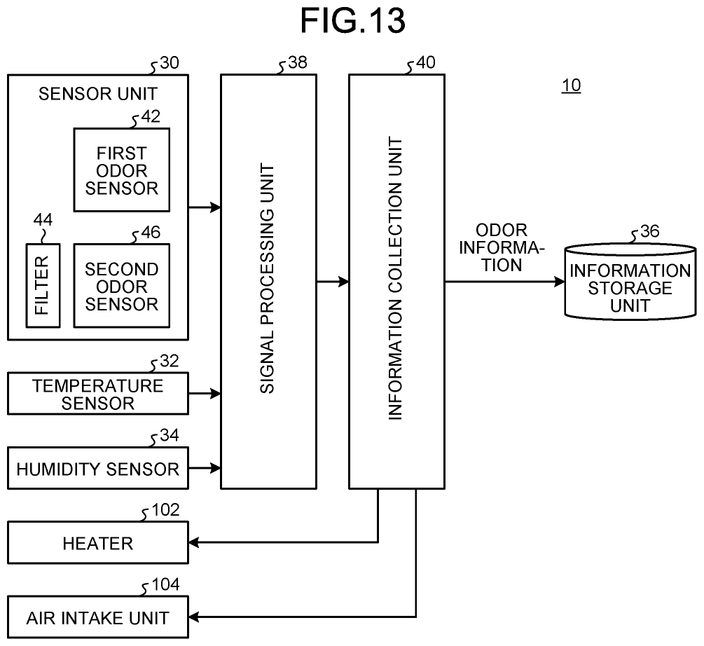

FIG. 13 is a diagram illustrating the configuration of the sensing system 10 according to the modification. The sensing system 10 according to the modification further includes a heater 102 and an air intake unit 104.

The heater 102 raises the temperature of the ambient air of the first odor sensor 42 and the second odor sensor 46. The air intake unit 104 is a fan, a pump, or the like. The air intake unit 104 ventilates the ambient air of the first odor sensor 42 and the second odor sensor 46. Specifically, the air intake unit 104 exhausts the ambient air of the first odor sensor 42 and the second odor sensor 46 to outside, and takes in fresh air from outside to the surroundings of the first odor sensor 42 and the second odor sensor 46.

In the modification, the collection controller 84 in the information collection unit 40 operates the heater 102 and the air intake unit 104 for a specific time after completing collection of the odor information. Then, after the specific time has elapsed, the collection controller 84 in the information collection unit 40 stops the operation of heater 102 and the operation of the air intake unit 104, and controls the first odor sensor 42 and the second odor sensor 46 to start detection of the amount of the odor-causing substance so as to collect new odor information.

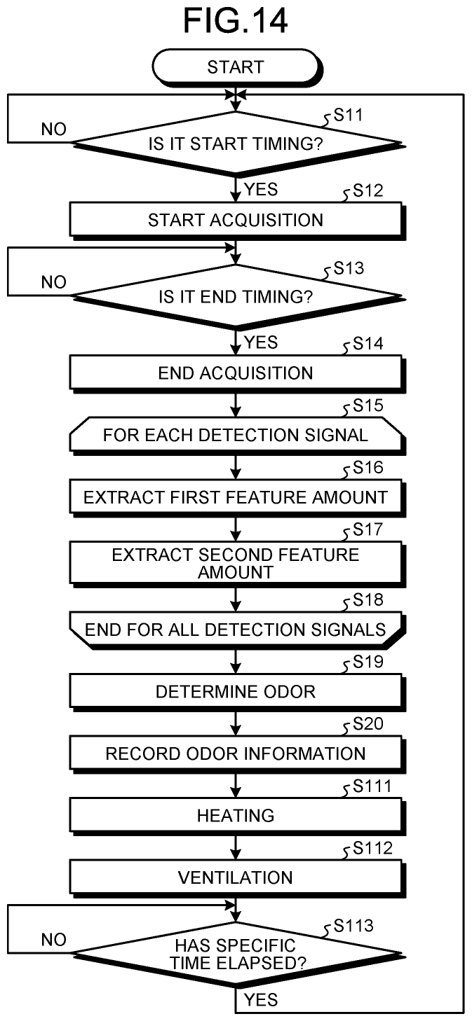

FIG. 14 is a flowchart illustrating a procedure of processing of the information collection unit 40 according to the modification. The information collection unit 40 according to the modification executes the processing in FIG. 14, for example. First, the information collection unit 40 according to the modification executes the processing similar to that of the processing described in FIG. 12 from S11 to S20.

After completing the process at S20, the information collection unit 40 advances the process to S111. At S111, the information collection unit 40 causes the heater 102 to operate. Thereby, the information collection unit 40 is able to raise the temperature of the ambient air of the first odor sensor 42 and the second odor sensor 46.

Subsequently, at S112, the information collection unit 40 causes the air intake unit 104 to operate. Thereby, the information collection unit 40 is able to ventilate the ambient air of the first odor sensor 42 and the second odor sensor 46.

Subsequently, at S113, the information collection unit 40 determines whether a specific time has elapsed. When the specific time has not elapsed (No at S113), the information collection unit 40 stands by for the process at S113. When the specific time has elapsed (Yes at S113), the information collection unit 40 returns the process to S11 and repeats the process from S11.

As described above, the sensing system 10 according to the modification raises the temperature of the ambient temperature of the first odor sensor 42 and the second odor sensor 46 and ventilates air every time an odor occurs and odor information is generated. Thereby, the sensing system 10 is able to remove odor-causing substances adsorbed to the first odor sensor 42 and the second odor sensor 46 and odor-causing substances floating in the surroundings, and resets the first odor sensor 42 and the second odor sensor 46 to the initial state. Therefore, even if odors repeatedly occur, the sensing system 10 according to the modification is able to collect the information regarding each of the odors with high accuracy.

The sensing system 10 according to the modification may include either one of the heater 102 and the air intake unit 104. Furthermore, the sensing system 10 according to the modification may not operate the heater 102 and the air intake unit 104 every time an odor occurs but may operate the heater 102 and the air intake unit 104 by each period determined in advance (for example, every one hour or the like), for example.

Hardware Configuration of Information Processing Apparatus 200

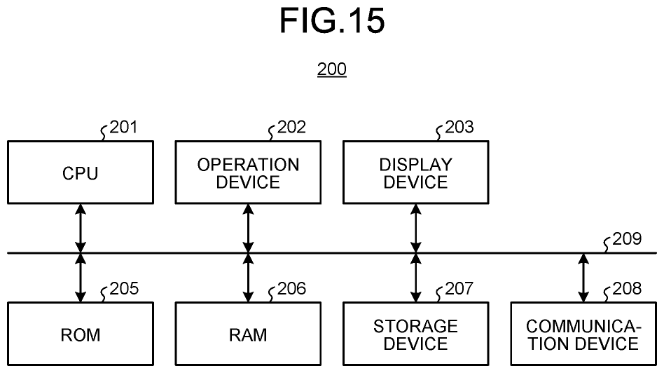

FIG. 15 is a diagram illustrating a hardware configuration of an information processing apparatus 200.

The signal processing unit 38 and the information collection unit 40 are implemented by the information processing apparatus 200 as illustrated in FIG. 15, for example. For example, one or more processors, as exemplified by CPU 102 together with RAM and/or ROM described below, can implement various functions, as described above, of the signal processing unit 38 and the information collection unit 40. The information processing apparatus 200 may have a hardware configuration similar to that of a general computer, for example. The information processing apparatus 200 includes a central processing unit (CPU) 201, an operation device 202, a display device 203, a read only memory (ROM) 205, a random access memory (RAM) 206, a storage device 207, a communication device 208, and a bus 209. The units are connected with each other over the bus 209.

The CPU 201 executes various kinds of processing through cooperation with various kinds of computer programs stored in the ROM 205 or the storage device 207 in advance by having a given area of the RAM 206 as a workspace to comprehensively control operation of each of the units configuring the information processing apparatus 200. The CPU 201 operates the operation device 202, the display device 203, the communication device 208, and the like through cooperation with computer programs stored in the ROM 205 or the storage device 207 in advance.

The operation device 202 is an input device such as a touch panel, a mouse, or a keyboard. The operation device 202 receives information input through an operation made by a user as an instruction signal, and outputs the instruction signal to the CPU 201. The display device 203 is a liquid crystal display (LCD) or the like, and displays various kinds of information based on display signals from the CPU 201.

The ROM 205 stores computer programs, various kinds of setting information, and the like for use in control of the information processing apparatus 200 in a non-rewritable manner. The RAM 206 is a volatile storage medium such as a synchronous dynamic random-access memory (SDRAM). The RAM 206 functions as the workspace of the CPU 201.

The storage device 207 is a rewritable recording device such as a semiconductor storage medium like a flash memory or a magnetically or optically recordable recording medium. The storage device 207 stores the computer programs for use in control of the information processing apparatus 200.

The communication device 208 transmits and receives data to and from the sensor unit 30. The communication device 208 may transmit and receive data to and from a server or the like over a network.

A computer program executed by the information processing apparatus 200 of the present embodiment is stored in a computer connected to a network such as the Internet and is provided by being downloaded over the network, for example. The computer program executed by the information processing apparatus 200 of the present embodiment may be provided by being recorded on a portable recording medium or the like in advance.

The computer program executed by the information processing apparatus 200 of the present embodiment has a module configuration including a first output signal acquisition module, a second output signal acquisition module, a temperature signal acquisition module, a humidity signal acquisition module, a difference calculation module, a correction module, a time generation module, a collection control module, a collection module, and a determination module. The CPU 201 (a processor) reads such a computer program from a storage medium or the like and loads each of the modules onto the RAM 206 (a main storage). Then, the CPU 201 (the processor) executes such a computer program to function as the first output signal acquisition unit 68, the second output signal acquisition unit 70, the temperature signal acquisition unit 72, the humidity signal acquisition unit 74, the difference calculation unit 76, the correction unit 78, the time generation unit 82, the collection controller 84, the collection unit 86, and the determination unit 90. Part or the whole of the first output signal acquisition unit 68, the second output signal acquisition unit 70, the temperature signal acquisition unit 72, the humidity signal acquisition unit 74, the difference calculation unit 76, the correction unit 78, the time generation unit 82, the collection controller 84, the collection unit 86, and the determination unit 90 may be configured by hardware. Furthermore, the RAM 206 or the storage device 207 functions as the information storage unit 36 and the odor pattern storage unit 88.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A sensing system comprising:
a first odor sensor including one or more detection elements respectively configured to detect an amount or amounts of an odor-causing substance or odor-causing substances existing in air, the one or more detection elements respectively outputting one or more first output signals;
a filter configured to remove the odor-causing substance existing in air;
a second odor sensor including one or more detection elements respectively configured to detect an amount or amounts of the odor-causing substance or substances existing in air that has passed through the filter, the one or more detection elements respectively outputting one or more second output signals; and
one or more processors that generate one or more detection signals by calculating a difference between the one or more first output signals from the one or more detection elements of the first odor sensor and the one or more second output signals from the one or more detection elements of the second odor sensor, the one or more processors generating odor information representing at least one of characteristics of the one or more detection signals based on one or more waveforms of the one or more detection signals and outputting the generated odor information.

2. The sensing system according to claim 1,
wherein the first odor sensor and the second odor sensor include a same type of the one or more detection elements, and
wherein the one or more processors calculate a difference between the first output signal and the second output signal, which are generated by the same type of the detection elements.

3. The sensing system according to claim 1, further comprising:
a temperature sensor configured to detect ambient temperature of the first odor sensor and the second odor sensor,
wherein the one or more processors correct the one or more detection signals based on the ambient temperature.

4. The sensing system according to claim 1, further comprising:
a humidity sensor configured to detect ambient humidity of the first odor sensor and the second odor sensor,
wherein the one or more processors correct the one or more detection signals based on the ambient humidity.

5. The sensing system according to claim 1, further comprising:
an air intake unit configured to take in air to surroundings of the first odor sensor and the second odor sensor,
wherein the one or more processors are configured to:
cause the air intake unit to operate for a given period,
cause the air intake unit to stop operation after the given period has elapsed, and
cause, after the stop of operation of the air intake unit, the first odor sensor and the second odor sensor to start detection of the amounts of the odor-causing substance.

6. The sensing system according to claim 1, further comprising:
a heater configured to raise a temperature of ambient air of the first odor sensor and the second odor sensor,
wherein the one or more processors are configured to
cause the heater to operate for a given period,
cause the heater to stop operation after the given period has elapsed, and
cause, after the stop of operation of the heater, the first odor sensor and the second odor sensor to start detection of the amount of the odor-causing substance.

7. The sensing system according to claim 1, wherein the one or more processors generate information representing the one or more waveforms of the one or more detection signals as at least a part of the odor information.

8. The sensing system according to claim 1,
wherein the one or more processors further detect, as a start timing, a timing at which the one or more detection signals starts to change, and
wherein the one or more processors generate the odor information based on the one or more detection signals in a given period from the start timing.

9. The sensing system according to claim 8, wherein the one or more processors detect, as the start timing, a timing at which any one of the one or more detection signals changes more greatly than a predetermined threshold value.

10. The sensing system according to claim 8,
wherein the one or more waveforms correspond to the one or more detection signals in a period from the start timing to a timing at which the given period has elapsed, and
wherein the one or more processors generate information representing the one or more waveforms as the odor information.

11. The sensing system according to claim 8,
wherein the one or more waveforms correspond to the one or more detection signals in a period from the start timing to a timing at which the given period has elapsed, and
wherein the one or more processors generate, as the odor information, a feature amount from the one or more waveforms.

12. The sensing system according to claim 8,
wherein the one or more waveforms correspond to the one or more detection signals in a period from the start timing to a first time later, and
wherein the one or more processors generate, as the odor information, a change amount of the one or more waveforms at an end point with respect to a start point.

13. The sensing system according to claim 8,
wherein the one or more waveforms correspond to the one or more detection signals in a period from the start timing to a second time later, and
wherein the one or more processors generate, as the odor information, a maximal value and a minimal value of the one or more waveforms.

14. The sensing system according to claim 8,
wherein the one or more processors determine a type of odor based on the one or more waveforms,
wherein the one or more waveforms correspond to the one or more detection signals in the given period from the start timing, and
wherein the one or more processors output, as the odor information, the determined type of odor.

15. The sensing system according to claim 8, wherein the one or more processors generates time of the start timing as at least a part of the odor information.

16. The sensing system according to claim 1,
wherein each of the one or more detection elements in each of the first and second odor sensors includes:
a quartz oscillator that is cut in a shape to oscillate by a piezoelectric effect; and
an adsorption film that is provided to the quartz oscillator to adsorb a given odor-causing substance, and
wherein each of the one or more first output signals and the one or more second output signals represents a change in a fundamental resonance frequency of the corresponding quartz oscillator.

17. An information processing apparatus comprising one or more processors configured to perform the following:
receiving one or more first output signals generated by one or more detection elements included in a first odor sensor, the one or more detection elements being respectively configured to detect an amount or amounts of an odor-causing substance or odor-causing substances existing in air,
receiving one or more second output signals generated by one or more detection elements included in a second odor sensor, the one or more detection elements being respectively configured to detect an amount or amounts of the odor-causing substance existing or substances in air that has passed through a filter configured to remove the odor-causing substance; and
calculating a difference between the one or more first output signals from the one or more detection elements of the first odor sensor and the one or more second output signals from the one or more detection elements of the second odor sensor so as to generate odor information representing at least one of characteristics of the one or more detection signals based on one or more waveforms of the one or more detection signal, and outputting the generated odor information.

18. A non-transitory computer-readable recording medium on which an executable program is recorded, the program instructing a computer to perform the following:
receiving one or more first output signals generated by one or more detection elements included in a first odor sensor, the one or more detection elements being respectively configured to detect an amount or amounts of an odor-causing substance or odor-causing substances existing in air,
receiving one or more second output signals generated by one or more detection elements included in a second odor sensor, the one or more detection elements being respectively configured to detect an amount or amounts of the odor-causing substance or substances existing in air that has passed through a filter configured to remove the odor-causing substance; and
calculating a difference between the one or more first output signals from the one or more detection elements of the first odor sensor and the one or more second output signals from the one or more detection elements of the second odor sensor so as to generate odor information representing at least one of characteristics of the one or more detection signals based on one or more waveforms of the one or more detection signal, and outputting the generated odor information.
